(19) United States
(12) Patent Application Publication
(10) Pub. No.: US 2019/0246875 A1
(43) Pub. Date: Aug. 15, 2019

(54) ENDOSCOPE SYSTEM AND ENDOSCOPE

(71) Applicant: OLYMPUS CORPORATION, Tokyo (JP)

(72) Inventors: Masakazu MIZOGUCHI, Tokyo (JP); Hideyuki KUGIMIYA, Tokyo (JP); Ryo KOSHIDA, Tokyo (JP)

(73) Assignee: OLYMPUS CORPORATION, Tokyo (JP)

(21) Appl. No.: 16/394,078

(22) Filed: Apr. 25, 2019

Related U.S. Application Data
(63) Continuation of application No. PCT/JP2017/037474, filed on Oct. 17, 2017.

(30) Foreign Application Priority Data
Oct. 31, 2016 (JP) 2016-213254

(51) Int. Cl. A61B 1/00; A61B 1/045; A61B 1/06; G02B 23/24; G06T 7/00

(52) U.S. Cl. CPC A61B 1/00009; A61B 1/045; A61B 1/0676; G06T 2207/10068; G06T 7/0012; A61B 1/00011; G02B 23/2484; G02B 23/2461

(57) ABSTRACT

An endoscope system includes: at least one image sensor configured to generate multiple pieces of image data in which acquisition areas of an object image are at least partially different from each other, or multiple pieces of image data having a disparity with regard to an identical object; a first processor configured to combine the pieces of image data to generate a single piece of combined image data; a second processor configured to execute image processing on the combined image data, and generate display image data to be presented on a display based on the combined image data on which the image processing has been executed. The second processor is disposed inside a predetermined casing, the first processor is disposed outside the predetermined casing, and the combined image data generated by the first processor is transmitted to the predetermined casing.

### CROSS-REFERENCE TO RELATED APPLICATION

This application is a continuation of PCT International Application No. PCT/JP2017/037474 filed on Oct. 17, 2017, which designates the United States, incorporated herein by reference, and which claims the benefit of priority from Japanese Patent Application No. 2016-213254, filed on Oct. 31, 2016, incorporated herein by reference.

### BACKGROUND

### 1. Technical Field

The present disclosure relates to an endoscope system and an endoscope.

### 2. Related Art

In recent years, there has been known image generation methods in which a disparity image is generated from two pieces of left-eye and right-eye image data that capture an object and have a disparity with respect to each other and the disparity image is presented in three dimensions on a display device. In endoscope systems used in medical fields, and the like, and having an endoscope and a processing device (processor) in a removable manner, there is a need to observe the observation target as a three-dimensional image so as to facilitate diagnosis and examination. As a technology to meet this need, there is a known endoscope that includes: an optical system that forms two optical paths for the left eye and the right eye; and two imaging elements that receive light from the respective optical paths for the left eye and the right eye from the optical system (for example, see Japanese Laid-open Patent Publication No. 9-080323). The processor having the endoscope attached thereto includes: signal processing circuitry that executes signal processing on each signal acquired from each imaging element; and image generation circuitry that generates a left-eye image and a right-eye image based on the signal having undergone signal processing.

### SUMMARY

In some embodiments, An endoscope system includes: at least one image sensor configured to generate multiple pieces of image data in which acquisition areas of an object image are at least partially different from each other, or multiple pieces of image data having a disparity with regard to an identical object; a first processor configured to combine the pieces of image data to generate a single piece of combined image data; a second processor configured to execute image processing on the combined image data, and generate display image data to be presented on a display based on the combined image data on which the image processing has been executed. The second processor is disposed inside a predetermined casing, the first processor is disposed outside the predetermined casing, and the combined image data generated by the first processor is transmitted to the predetermined casing.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram that illustrates a schematic configuration of an endoscope system according to a first embodiment of the disclosure;

FIG. 2 is a block diagram that illustrates a schematic configuration of the endoscope system according to the first embodiment of the disclosure;

FIG. 3 is a diagram that illustrates an example of a combined image that is combined by an image combining unit in the endoscope system according to the first embodiment of the disclosure;

FIG. 4 is a diagram that illustrates another example of the combined image that is combined by the image combining unit in the endoscope system according to the first embodiment of the disclosure;

FIG. 5 is a block diagram that illustrates a schematic configuration of an endoscope system according to a modification 1 of the first embodiment of the disclosure;

FIG. 6 is a block diagram that illustrates a schematic configuration of an endoscope system according to a modification 2 of the first embodiment of the disclosure;

FIG. 7 is a block diagram that illustrates a schematic configuration of an endoscope system according to a second embodiment of the disclosure;

FIG. 8 is a block diagram that illustrates a schematic configuration of an endoscope system according to a modification of the second embodiment of the disclosure;

FIG. 9 is a block diagram that illustrates a schematic configuration of an endoscope system according to a third embodiment of the disclosure;

FIG. 10 is a diagram that illustrates a schematic configuration of an endoscope system according to a fourth embodiment of the disclosure; and

FIG. 11 is a block diagram that illustrates a schematic configuration of an endoscope system according to the fourth embodiment of the disclosure.

### DETAILED DESCRIPTION

An aspect (hereinafter referred to as “embodiment”) for carrying out the disclosure is explained below. In the embodiment, as an example of the endoscope system according to the disclosure, an explanation is given of a medical endoscope system that captures and displays images inside the body of the subject, such as patient. The disclosure is not limited to the embodiment. Furthermore, in the description of drawings, the same components are attached with the same reference numeral and are explained.

### First Embodiment

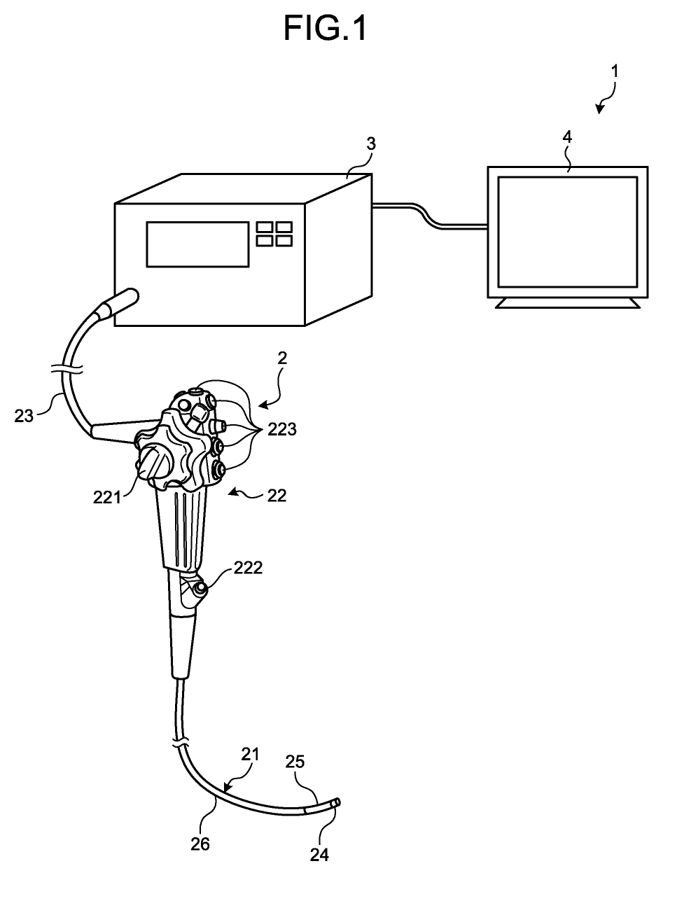

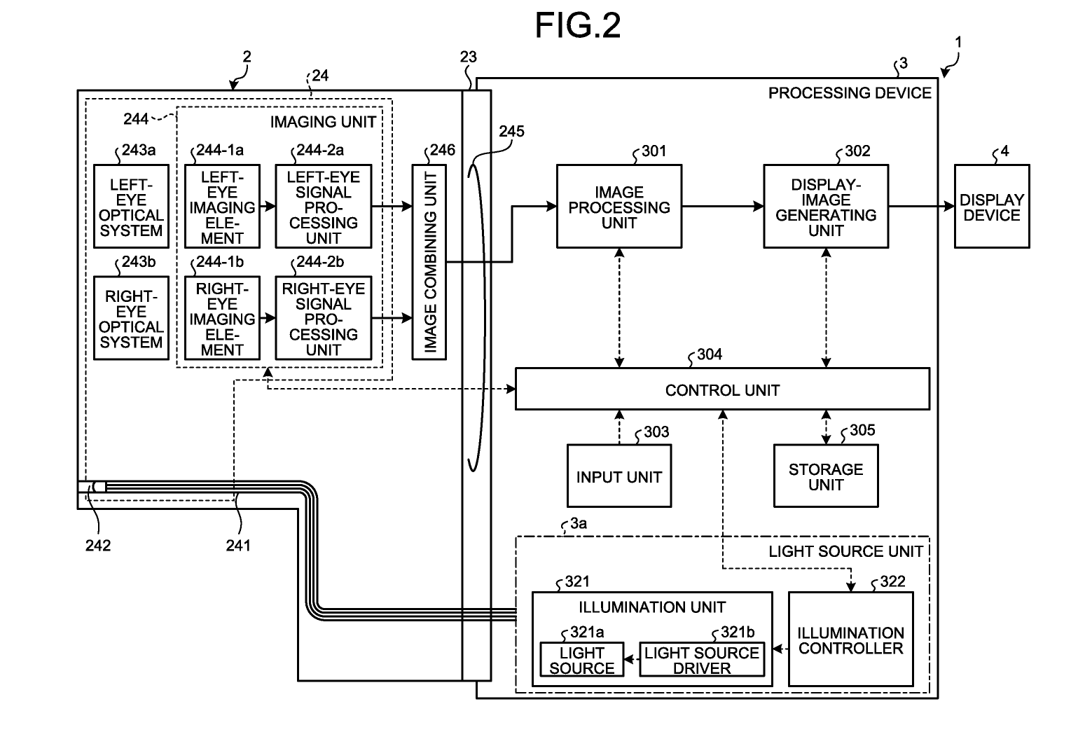

FIG. 1 is a diagram that illustrates a schematic configuration of an endoscope system according to a first embodiment of the disclosure. FIG. 2 is a block diagram that illustrates a schematic configuration of the endoscope system according to the first embodiment.

An endoscope system 1 illustrated in FIG. 1 and FIG. 2 includes: an endoscope 2 that captures images (hereinafter, also referred to as endoscope images) inside the body of the subject by inserting an distal end portion thereof into the body of the subject; a processing device 3 that includes a light source unit 3a that generates illumination light to be output from the distal end of the endoscope 2, performs predetermined signal processing on image signals captured by the endoscope 2, and integrally controls the overall operation of the endoscope system 1; and a display device 4 that displays endoscope images generated during signal processing by the processing device 3. In FIG. 2, the arrow in a solid line indicates transmission of electric signals regarding an image, and the arrow in a dashed line indicates transmission of electric signals regarding a control.

The endoscope 2 includes: an insertion portion 21 having flexibility and formed in an elongated shape; an operating portion 22 that is connected to the proximal end side of the insertion portion 21 and receives inputs of various operating signals; and a universal code 23 that extends in a direction different from the direction in which the insertion portion 21 extends from the operating portion 22 and has various built-in cables connected to the processing device 3 (including the light source unit 3a).

The insertion portion 21 includes: a distal end portion 24 having a built-in imaging unit 244, in which pixels are arranged in two dimensions to receive light and conduct photoelectric conversion so as to generate signals; a curved portion 25 that is composed of multiple curved pieces and is flexible; and a flexible tube portion 26 having flexibility, formed in an elongated shape, and connected to the proximal end side of the curved portion 25. The insertion portion 21 is inserted into the body cavity of the subject and uses the imaging unit 244 to capture the object, such as living tissue, located at a position out of range of outside light.

The distal end portion 24 includes: a light guide 241 that is configured by using glass fibers, or the like, and forms a light guide path for light generated by the light source unit 3a; an illumination lens 242 provided at the distal end of the light guide 241; a left-eye optical system 243a and a right-eye optical system 243b for focusing; the imaging unit 244 that receives the light, focused by the left-eye optical system 243a and the right-eye optical system 243b, conducts photoelectric conversion into electric signals, and executes predetermined signal processing; and an image combining unit 246 that combines two pieces of image data acquired by the imaging unit 244 via the left-eye optical system 243a and the right-eye optical system 243b to generate one piece of combined image data.

The left-eye optical system 243a is configured by using one or more lenses and disposed at the former stage of the imaging unit 244 to focus the incident light from the object. The left-eye optical system 243a may have an optical zoom function for changing the angle of view and a focus function for changing the focal point.

The right-eye optical system 243b is configured by using one or more lenses and disposed at the former stage of the imaging unit 244 to focus the incident light from the object with a disparity with respect to the left-eye optical system 243a. The right-eye optical system 243b may have an optical zoom function for changing the angle of view and a focus function for changing the focal point.

The imaging unit 244 includes a left-eye imaging element 244-1a, a right-eye imaging element 244-1b, a left-eye signal processing unit 244-2a, and a right-eye signal processing unit 244-2b.

The left-eye imaging element 244-1a conducts photoelectric conversion on light from the left-eye optical system 243a in accordance with a control signal received from the processing device 3 and generates electric signals (left-eye image signals) corresponding to one frame forming a single image. Specifically, in the left-eye imaging element 244-1a, pixels are arranged in a matrix, each including a photo diode that stores the electric charge corresponding to the amount of light, a capacitor that converts the electric charge transferred from the photo diode into a voltage level, and the like, each of the pixels conducts photoelectric conversion on light from the left-eye optical system 243a to generate an electric signal, the electric signal generated by the pixel, which is optionally set as the target for reading out of the pixels, is sequentially read, and it is output as an image signal. For the left-eye imaging element 244-1a, an exposure process is controlled in accordance with a control signal received from the processing device 3. A color filter is provided on the light receiving surface of the left-eye imaging element 244-1a so that each pixel receives light in any one of the wavelength bands of color components, red (R), green (G), and blue (B).

The right-eye imaging element 244-1b conducts photoelectric conversion on light from the right-eye optical system 243b in accordance with a control signal received from the processing device 3 and generates electric signals (right-eye image signals) corresponding to one frame forming a single image. Specifically, in the right-eye imaging element 244-1b, pixels are arranged in a matrix, each including a photo diode that stores the electric charge corresponding to the amount of light, a capacitor that converts the electric charge transferred from the photo diode into a voltage level, and the like, each of the pixels conducts photoelectric conversion on light from the right-eye optical system 243b to generate an electric signal, the electric signal generated by the pixel, which is optionally set as the target for reading out of the pixels, is sequentially read, and it is output as an image signal. For the right-eye imaging element 244-1b, an exposure process is controlled in accordance with a control signal received from the processing device 3. A color filter is provided on the light receiving surface of the right-eye imaging element 244-1b so that each pixel receives light in any one of the wavelength bands of color components, red (R), green (G), and blue (B).

The left-eye imaging element 244-1a and the right-eye imaging element 244-1b are implemented by using, for example, a CCD (Charge Coupled Device) image sensor or a CMOS (Complementary Metal Oxide Semiconductor) image sensor. Furthermore, each of the left-eye imaging element 244-1a and the right-eye imaging element 244-1b may be configured by using a single image sensor, or it may be configured by using multiple image sensors, for example, three image sensors.

A left-eye image obtained by the left-eye imaging element 244-1a and a right-eye image obtained by the right-eye imaging element 244-1b are images which capture the identical object, in which acquisition areas of an object image are at least partially different from each other, and which have a disparity. When the angles of the optical axes of the left-eye optical system 243a and the right-eye optical system 243b relative to the object are different from each other, the acquisition areas of object images (parts that appear as images) are also different from each other.

The left-eye signal processing unit 244-2a performs analog processing for executing noise removal processing or clamp processing or A/D conversion processing on left-eye image data (analog) output from the left-eye imaging element 244-1a and outputs left-eye image data (digital) including a left-eye image to the image combining unit 246.

The right-eye signal processing unit 244-2b performs analog processing for executing noise removal processing or clamp processing or A/D conversion processing on right-eye image data (analog) output from the right-eye imaging element 244-1b and outputs right-eye image data (digital) including a right-eye image to the image combining unit 246.

The operating portion 22 includes: a curved knob 221 for curving the curved portion 25 in a vertical direction and in a horizontal direction; a treatment-tool insertion portion 222 through which a treatment tool, such as biopsy forceps, electric cautery, or examination probe, is inserted into the body cavity of the subject; and a plurality of switches 223 that is an operation input unit that inputs operation command signals from, in addition to the processing device 3, an air supply unit, a water supply unit, or a peripheral device for screen-display control, and the like. A treatment tool inserted through the treatment-tool insertion portion 222 is protruded from an opening section (not illustrated) via a treatment-tool channel (not illustrated) at the distal end portion 24.

The image combining unit 246 receives left-eye image data and right-eye image data representing an endoscope image generated by the imaging unit 244. The image combining unit 246 combines received left-eye image data and right-eye image data to generate one piece of combined image data. The image combining unit 246 outputs the generated combined image data to the processing device 3. The image combining unit 246 is provided in, for example, the operating portion 22. Alternatively, the image combining unit 246 may be provided in the distal end portion 24 or the connector portion of the universal code 23. The image combining unit 246 is configured by using a general-purpose processor such as a CPU (Central Processing Unit), or a dedicated processor such as various arithmetic circuits to perform a specific function, e.g., ASIC (Application Specific Integrated Circuit), or FPGA (Field Programmable Gate Array) that is a programmable logic device in which processing details are rewritable.

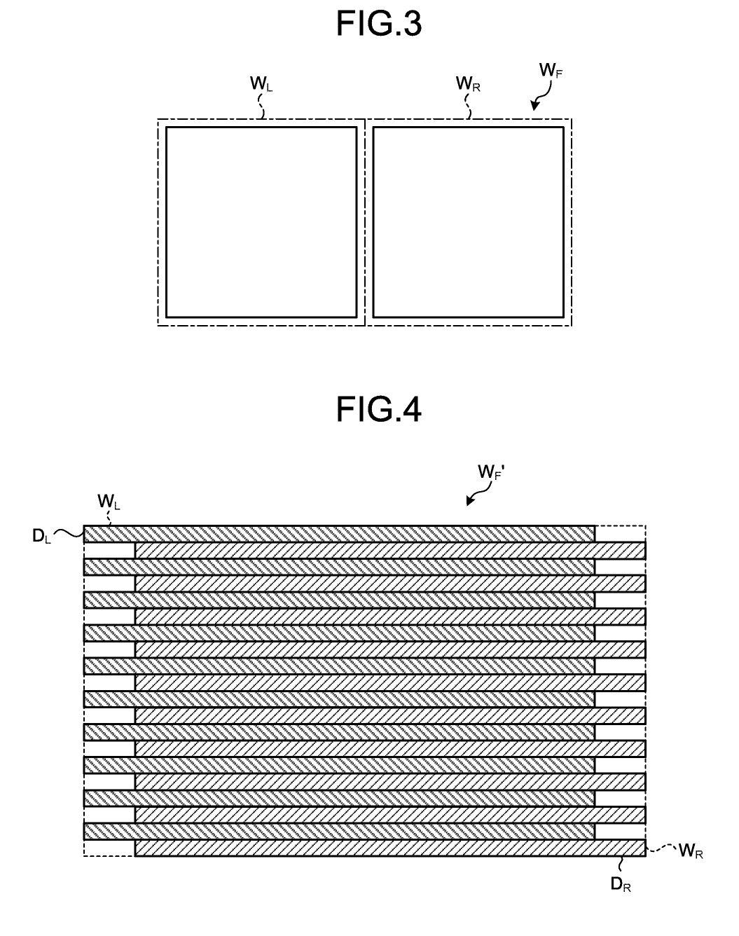

FIG. 3 is a diagram that illustrates an example of a combined image that is combined by the image combining unit in the endoscope system according to the first embodiment of the disclosure. As illustrated in FIG. 3, the image combining unit 246 arranges a left-eye image W_{L} and a right-eye image W_{R} side by side to combine and generate one piece of combined image W_{F}. For the combined image W_{F}, the left-eye image W_{L} and the right-eye image W_{R} may be arranged such that the horizontal lines of pixel arrays are aligned (see for example FIG. 3) or the left-eye image W_{L} and the right-eye image W_{R} may be arranged such that the vertical lines are aligned. Here, the left-eye image W_{L} and the right-eye image W_{R} are images that include pixel values in an optical black region, and the like, other than valid pixel regions.

Instead of arranging the left-eye image W_{L} and the right-eye image W_{R} as described above, the left-eye image W_{L} and the right-eye image W_{R} may be periodically arranged line by line. FIG. 4 is a diagram that illustrates another example of the combined image that is combined by the image combining unit in the endoscope system according to the first embodiment of the disclosure. As illustrated in FIG. 4, the image combining unit 246 generates a combined image W_{F}′ by periodically arranging a line image D_{L} in a horizontal line of the left-eye image W_{L} and a line image D_{R} in a horizontal line of the right-eye image W_{R} while shifting them by the specified shift amount. Specifically, the image combining unit 246 alternately arranges the line image D_{L} in the odd line included in the left-eye image W_{L} and the line image D_{R} in the even line included in the right-eye image W_{R} while shifting them by the specified shift amount. The above-described combined image W_{F}′ is also called a line-by-line image. The horizontal line mentioned here corresponds to a line formed by pixels arranged along one array direction in the imaging element in which a plurality of pixels is arranged in a matrix. Furthermore, the combined image W_{F}′ may be an image in which the line image D_{L} of the left-eye image W_{L} and the line image D_{R} of the right-eye image W_{R} are alternately arranged by the shifted amount of zero, i.e., an image in which both ends of the line image D_{L} and the line image D_{R} are aligned, as long as the left-eye image W_{L} and the right-eye image W_{R} may be combined into a single piece of data. Moreover, the image combining unit 246 may generate a combined image by periodically arranging by vertical lines that are lines vertical to the horizontal lines.

The universal code 23 has built-in at least the light guide 241 and a group cable 245 that combines one or more signal lines. The group cable 245 includes a signal line for transmitting image signals, a signal line for transmitting control signals to control the imaging unit 244, and a signal line for transmitting and receiving information including unique information, and the like, related to the endoscope 2 (the imaging unit 244). In explanation according to the present embodiment, electric signals are transmitted by using a signal line; however, optical signals may be transmitted, or signals may be transmitted between the endoscope 2 and the processing device 3 via radio communications.

Furthermore, the endoscope 2 includes a memory (not illustrated) that stores information about the endoscope 2. The memory stores identification information indicating the type and the model number of the endoscope 2, the type of the left-eye imaging element 244-1a, the right-eye imaging element 244-1b, and the like. Here, the memory may store various parameters for image processing on image data captured by the left-eye imaging element 244-1a and the right-eye imaging element 244-1b, e.g., parameter for white balance (WB) adjustment.

When the endoscope 2 is attached to the processing device 3, the above-described information on the endoscope 2 is output to the processing device 3 during communication processing with the processing device 3. Alternatively, there is a case where a connection pin is provided in a connector in accordance with the rule that corresponds to the information on the endoscope 2 and the processing device 3 recognizes the connection of the endoscope 2 based on the connection state between the connection pin at the side of the processing device 3 and the connection pin at the side of the endoscope 2 when the endoscope 2 is attached.

Next, the configuration of the processing device 3 is explained. The processing device 3 includes an image processing unit 301, a display-image generating unit 302, an input unit 303, a control unit 304, and a storage unit 305.

The image processing unit 301 calculates the pixel value of a luminance component (e.g., the Y component in YCrCb) and the pixel value of each color component, RGB, at each pixel location in each of the left-eye image and the right-eye image based on the received combined image and executes signal processing, such as pixel defect correction, optical correction, color correction, optical black subtraction, noise reduction, white balance adjustment, or interpolation processing, on the left-eye image and the right-eye image. Pixel defect correction is to assign the pixel value of a defect pixel based on the pixels values of the surrounding pixels of the defect pixel. Optical correction is to correct optical distortions, or the like, of a lens. Color correction is to correct a color temperature or correct color deviation.

Furthermore, the image processing unit 301 performs zoom processing or enhancement processing on a combined image having undergone the above-described image processing in accordance with the setting input via the input unit 303. Specifically, the image processing unit 301 performs enhancement processing to enhance the R component when, for example, the setting indicating that the red color component is to be enhanced has been made via the input unit 303.

The display-image generating unit 302 generates a synthetic image by synthesizing the background image including the display area of an endoscope image with the textual information regarding the endoscope image. Specifically, the display-image generating unit 302 refers to the storage unit 305, superimposes the textual information, or the like, regarding the captured endoscope image on the background image, e.g., black background, forming the display screen, and synthesizes them.

After generating the synthetic image having undergone the above-described synthesis process, the display-image generating unit 302 executes signal processing to obtain signals in a format displayable on a display device 4 and generates image signals for display. Specifically, the display-image generating unit 302 first acquires the left-eye image and the right-eye image of the combined image from the image processing unit 301 and generates a disparity image, what is called side-by-side image, in which the left-eye image and the right-eye image are located at positions apart from each other and at positions to generate a disparity. Then, the display-image generating unit 302 superimposes the generated disparity image on the image forming the display screen, performs a compression process, or the like, on the image signals including the image, and generates the image signals for display. The display-image generating unit 302 transmits the generated image signals for display to the display device 4. It is possible to use not only side-by-side images but also, for example, line-by-line images that are combined by alternately arranging line data in a left-eye image and line data in a right-eye image while shifting them by the amount of shift to generate a disparity.

The image processing unit 301 and the display-image generating unit 302 are configured by using a general-purpose processor such as CPU or a dedicated processor such as various arithmetic circuits to execute a specific function, e.g., ASIC or FPGA.

The input unit 303 is implemented by using a keyboard, mouse, switch, or touch panel to receive inputs of various signals such as operation command signals for giving commands to operate the endoscope system 1. Furthermore, the input unit 303 may include the switch 223 provided in the operating portion 22 or a portable terminal such as external tablet-type computer.

The control unit 304 is configured by using a general-purpose processor such as CPU or a dedicated processor such as various arithmetic circuits to perform a specific function, e.g., ASIC, and it controls, for example, driving of each component including the imaging unit 244 and the light source unit 3a or controls input/output of information to and from each component. The control unit 304 transmits control information data (e.g., read timing) for capturing control, stored in the storage unit 305, as control signals to the imaging unit 244 via a predetermined signal line included in the group cable 245. Furthermore, the control unit 304 controls the display device 4 so as to display the image corresponding to image signals for display, generated by the display-image generating unit 302. The control unit 304 is configured by using a general-purpose processor such as CPU or a dedicated processor such as various arithmetic circuits to perform a specific function, e.g., ASIC.

The storage unit 305 stores data including various programs for operating the endoscope system 1, various parameters needed to operate the endoscope system 1, and the like, and information regarding the synthesis process, what is called on-screen display (OSD) process, to generate synthetic images by superimposing image information having undergone predetermined image processing on textual information related to the image information. The textual information is information indicating patient information, device information, examination information, or the like. Furthermore, the storage unit 305 stores identification information on the processing device 3. Here, the identification information includes unique information (ID), model year, and specs information, or the like, of the processing device 3.

Furthermore, the storage unit 305 stores various programs including an image-acquisition processing program to implement an image-acquisition processing method of the processing device 3. Various programs may be widely distributed by being recorded in a recording medium readable by a computer, such as hard disk, flash memory, CD-ROM, DVD-ROM, or flexible disk. The above-described various programs are available by being downloaded via a communication network. The communication network mentioned here is provided by, for example, the existing public networks, LAN (Local Area Network), or WAN (Wide Area Network) regardless of whether it is wired or wireless.

Furthermore, the storage unit 305 stores information regarding the background image forming a display image and regarding the synthesis process, what is called on-screen display (OSD) process, to generate synthetic images by superimposing textual information related to information on an endoscope image, or the like, on the background image. The textual information is information indicating patient information, device information, examination information, or the like.

The storage unit 305 having the above-described configuration is implemented by using a ROM (Read Only Memory) having previously installed various programs, and the like, a RAM (Random Access Memory) storing calculation parameters, data, and the like, for each process, a hard disk, or the like.

Next, the configuration of the light source unit 3a is explained. The light source unit 3a includes an illumination unit 321 and an illumination controller 322. Under the control of the illumination controller 322, the illumination unit 321 outputs illumination light. The illumination unit 321 includes a light source 321a and a light source driver 321b.

The light source 321a is configured by using an LED light source that outputs white light, one or more lenses, and the like, and it outputs light (illumination light) due to driving of the LED light source. The illumination light generated by the light source 321a is output toward the object from the distal end of the distal end portion 24 via the light guide 241. Furthermore, the light source 321a is implemented by using any one of an LED light source, laser light source, xenon lamp, halogen lamp, and the like.

Under the control of the illumination controller 322, the light source driver 321b supplies currents to the light source 321a, thereby outputting illumination light to the light source 321a.

Based on control signals (light control signals) from the control unit 304, the illumination controller 322 controls the amount of power supplied to the light source 321a and controls the drive timing of the light source 321a. The illumination controller 322 is configured by using a general-purpose processor such as CPU or a dedicated processor such as various arithmetic circuits to perform a specific function, e.g., ASIC.

The display device 4 presents a display image corresponding to the image signal received from the processing device 3 (the display-image generating unit 302) via a video cable. The display device 4 is configured by using a liquid crystal or organic EL (Electro Luminescence) monitor, or the like.

The user observes a disparity image displayed on the display device 4 with the glasses having polarization properties. This allows the user to observe a three-dimensional image by observing the left-eye image with the left eye and observing the right-eye image with the right eye.

According to the first embodiment of the disclosure described above, the image combining unit 246 provided in the endoscope 2 combines the left-eye image data and the right-eye image data representing the endoscope image generated by the imaging unit 244 to generate one piece of combined image data and outputs it to the processing device 3. Thus, the processing device 3 only has to generate a disparity image for display by executing image processing on a single piece of combined image data; as a result, it is possible to reduce the size of circuitry in the processing device to generate a disparity image by using a right-eye image and a left-eye image.

Modification 1 of the First Embodiment

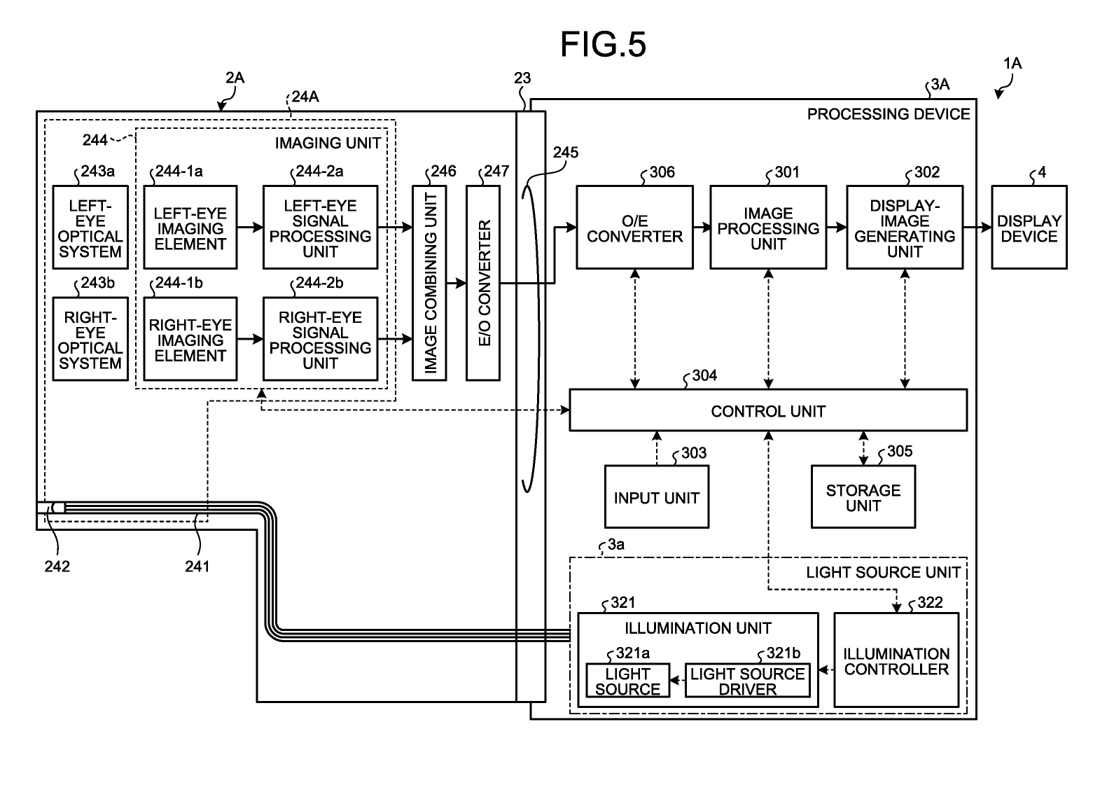

According to a modification 1, combined image data generated by the image combining unit 246 is converted into optical signals and transmitted to a processing device 3A. FIG. 5 is a block diagram that illustrates a schematic configuration of an endoscope system according to the modification 1 of the first embodiment of the disclosure.

An endoscope system 1A illustrated in FIG. 5 includes: an endoscope 2A that captures images inside the body of the subject by inserting the distal end portion into the body of the subject; the processing device 3A that includes the light source unit 3a that generates illumination light to be output from the distal end of the endoscope 2A, executes predetermined signal processing on image signals captured by the endoscope 2A, and controls the overall operation of the endoscope system 1A in an integrated manner; and the display device 4 that displays endoscope images generated during signal processing by the processing device 3A. The configuration different from that in the above-described first embodiment is explained below.

The endoscope 2A includes a distal end portion 24A instead of the distal end portion 24 in the above-described configuration of the endoscope 2. The distal end portion 24A includes an E/O converter 247 in addition to the light guide 241, the illumination lens 242, the left-eye optical system 243a and the right-eye optical system 243b, the imaging unit 244, and the image combining unit 246 described above.

The E/O converter 247 converts combined image data, which is digital signals generated by the image combining unit 246, into optical signals and outputs them to the processing device 3A. The E/O converter 247 is configured by using, for example, a laser diode (LD). Under the control of the control unit 304, the laser diode outputs laser light (optical signal) including the combined image data as pixel information to the processing device 3A.

The processing device 3A includes an O/E converter 306 in addition to the image processing unit 301, the display-image generating unit 302, the input unit 303, the control unit 304, and the storage unit 305 described above.

The O/E converter 306 receives optical signals including the pixel information output from the E/O converter 247 and converts them into electric signals. The O/E converter 306 is configured by using a photo diode (PD) that receives light (receives optical signals) output from the E/O converter 247. Furthermore, the E/O converter 247 and the O/E converter 306 are coupled to each other with optical fibers. The subsequent processes to generate and present display images are the same as those in the above-described first embodiment. Control signals may be transmitted and received via a signal line or may be transmitted and received after being converted into optical signals.

In the case where combined image data is transmitted to the processing device 3A through optical communications as in the modification 1, too, the advantage similar to that in the above-described first embodiment may be obtained.

In explanation according to the above-described modification 1, the processing device 3A includes the O/E converter 306; however, for example, an O/E converter may be provided in a connector connected to the processing device 3A of the endoscope 2A.

Modification 2 of the First Embodiment

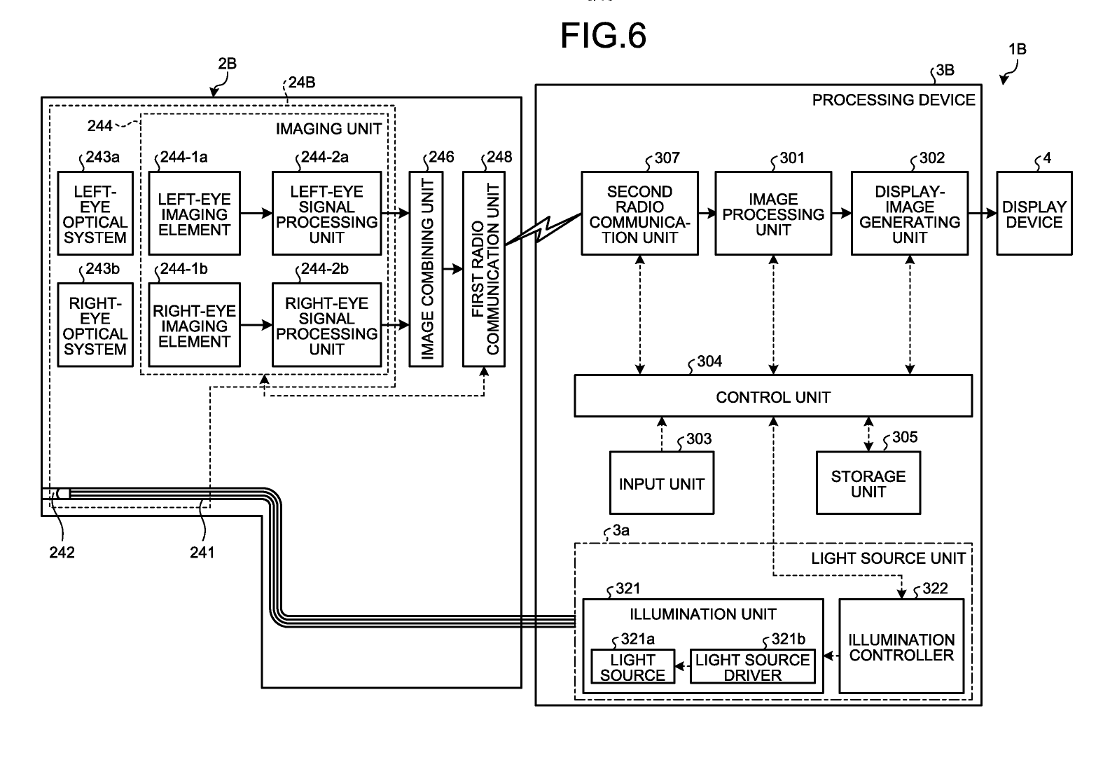

According to a modification 2, combined image data generated by the image combining unit 246 is transmitted to a processing device 3B via radio communications. FIG. 6 is a block diagram that illustrates a schematic configuration of an endoscope system according to the modification 2 of the first embodiment of the disclosure.

An endoscope system 1B illustrated in FIG. 6 includes: an endoscope 2B that captures images inside the body of the subject by inserting the distal end portion into the body of the subject; the processing device 3B that includes the light source unit 3a that generates illumination light to be output from the distal end of the endoscope 2B, executes predetermined signal processing on image signals captured by the endoscope 2B, and controls the overall operation of the endoscope system 1B in an integrated manner; and the display device 4 that displays endoscope images generated during signal processing by the processing device 3B. The configuration different from that in the above-described first embodiment is explained below.

The endoscope 2B includes a distal end portion 24B instead of the distal end portion 24 in the above-described configuration of the endoscope 2. The distal end portion 24B includes a first radio communication unit 248 in addition to the light guide 241, the illumination lens 242, the left-eye optical system 243a and the right-eye optical system 243b, the imaging unit 244, and the image combining unit 246 described above.

The first radio communication unit 248 superimposes combined image data, which is a digital signal generated by the image combining unit 246, on a radio signal and transmits them to an external unit. The first radio communication unit 248 is configured by using an antenna that is capable of transmitting radio signals to an external unit. The first radio communication unit 248 may use digital radio waves or analog radio waves. Furthermore, the first radio communication unit 248 outputs control signals acquired from the processing device 3B to the imaging unit 244.

The processing device 3B includes a second radio communication unit 307 in addition to the image processing unit 301, the display-image generating unit 302, the input unit 303, the control unit 304, and the storage unit 305 described above.

The second radio communication unit 307 receives radio signals transmitted from the first radio communication unit 248. The second radio communication unit 307 is configured by using an antenna that is capable of receiving radio signals.

In the case where combined image data is transmitted to the processing device 3B through radio communications as in the modification 2, too, the advantage similar to that in the above-described first embodiment may be obtained.

Furthermore, in the above-described modification 2, the endoscope 2B and the light source unit 3a are coupled to each other via a cable through which the light guide 241 is inserted. Control signals may be transmitted and received via a signal line, may be transmitted and received via radio communications, or may be transmitted and received after being converted into optical signals.

### Second Embodiment

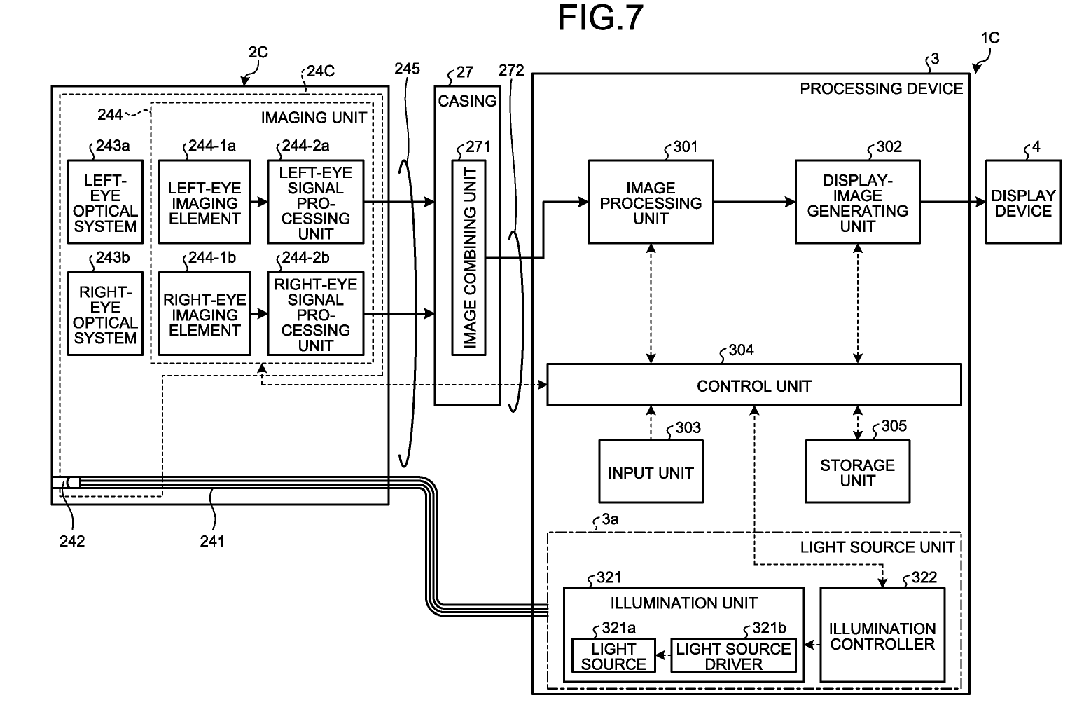

Next, a second embodiment of the disclosure is explained with reference to FIG. 7. According to the second embodiment, an image combining unit, which combines a right-eye image and a left-eye image, is disposed in a casing 27 that electrically connects an endoscope 2C and the processing device 3. FIG. 7 is a block diagram that illustrates a schematic configuration of an endoscope system according to the second embodiment of the disclosure.

An endoscope system 1C illustrated in FIG. 7 includes: an endoscope 2C that captures images inside the body of the subject by inserting the distal end portion into the body of the subject; the processing device 3 that includes the light source unit 3a that generates illumination light to be output from the distal end of the endoscope 2C, executes predetermined signal processing on image signals captured by the endoscope 2C, and controls the overall operation of the endoscope system 1C in an integrated manner; and the display device 4 that displays endoscope images generated during signal processing by the processing device 3. The configuration different from that in the above-described first embodiment is explained below.

The endoscope 2C includes a distal end portion 24C instead of the distal end portion 24 in the above-described configuration of the endoscope 2. The distal end portion 24C includes the light guide 241, the illumination lens 242, the left-eye optical system 243a and the right-eye optical system 243b, and the imaging unit 244 described above.

Information is transmitted and received between the endoscope 2C and the processing device 3 via the casing 27. One side of the casing 27 is electrically coupled to the endoscope 2C and the other side thereof is electrically coupled to the processing device 3. The casing 27 is coupled to the endoscope 2C via the group cable 245 and is coupled to the processing device 3 via a group cable 272. The casing 27 includes an image combining unit 271.

In the same manner as the image combining unit 246, the image combining unit 271 receives, from the endoscope 2C, left-eye image data and right-eye image data representing the endoscope image generated by the imaging unit 244. The image combining unit 271 combines the received left-eye image data and right-eye image data to generate one piece of combined image data. The image combining unit 271 outputs the generated combined image data to the processing device 3. In the same manner as in the above-described first embodiment, the processing device 3 generates a disparity image to be displayed on the display device 4 based on the received combined image data. The image combining unit 271 is configured by using a general-purpose processor such as CPU or a dedicated processor such as various arithmetic circuits to perform a specific function, e.g., ASIC.

In the case where the endoscope 2C and the processing device 3 are electrically coupled to each other through the casing 27, combined image data is generated by the image combining unit 271 provided in the casing 27, and the generated combined image data is transmitted to the processing device 3 as in the second embodiment, too, the advantage similar to that in the above-described first embodiment may be obtained.

Modification of the Second Embodiment

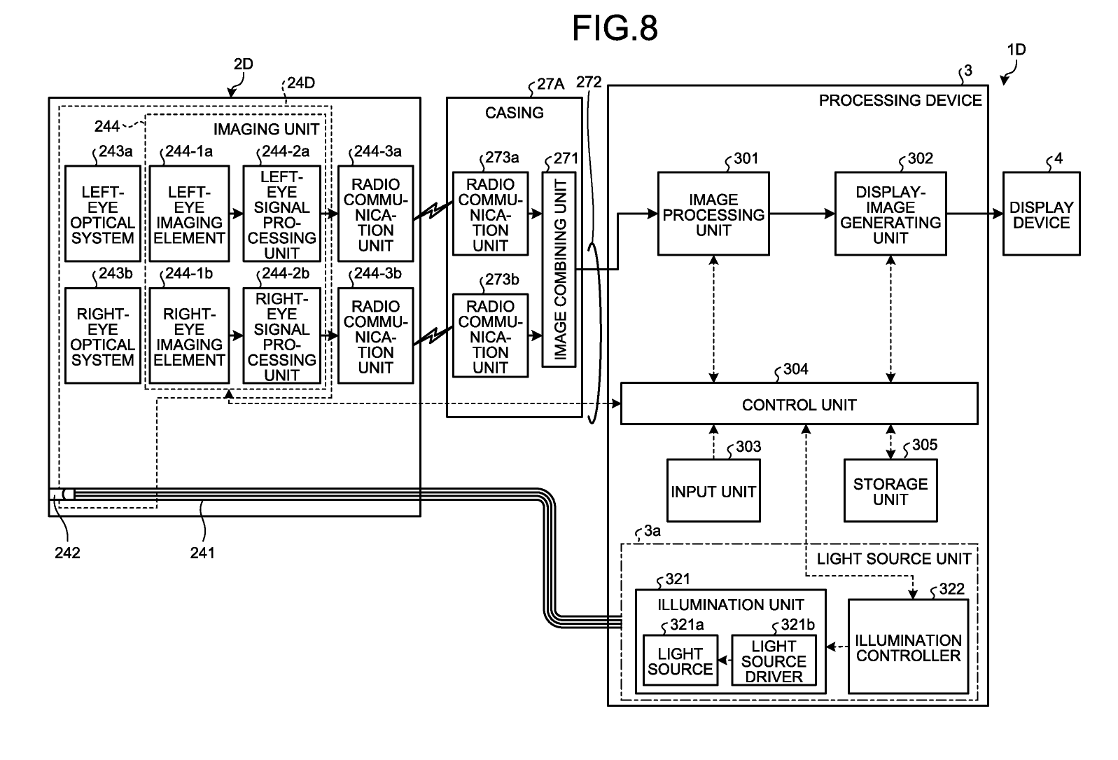

According to the present modification, left-eye image data and right-eye image data generated by the imaging unit 244 are transmitted to the casing via radio communications. FIG. 8 is a block diagram that illustrates a schematic configuration of an endoscope system according to the modification of the second embodiment of the disclosure.

An endoscope system 1D illustrated in FIG. 8 includes: an endoscope 2D that captures images inside the body of the subject by inserting the distal end portion into the body of the subject; the processing device 3 that includes the light source unit 3a that generates illumination light to be output from the distal end of the endoscope 2D, executes predetermined signal processing on image signals captured by the endoscope 2D, and controls the overall operation of the endoscope system 1D in an integrated manner; and the display device 4 that displays endoscope images generated during signal processing by the processing device 3. Furthermore, information is transmitted and received between the endoscope 2D and the processing device 3 via a casing 27A. The configuration different from that in the above-described second embodiment is explained below.

The endoscope 2D further includes radio communication units 244-3a, 244-3b in the above-described configuration of the endoscope 2C. Furthermore, in explanation, a distal end portion 24D has the same configuration as that of the above-described distal end portion 24C and outputs generated image data to the radio communication units 244-3a, 244-3b. The radio communication units 244-3a, 244-3b constitute a first radio communication unit.

The casing 27A includes the above-described image combining unit 271 and radio communication units 273a, 273b. One side of the casing 27A is capable of performing radio communications with the endoscope 2D, and the other side thereof is coupled to the processing device 3 via the group cable 272. The radio communication units 273a, 273b constitute a second radio communication unit.

According to the present modification, the radio communication units 244-3a, 244-3b superimpose left-eye image data and right-eye image data, which represent an endoscope image generated by the imaging unit 244, on radio signals and transmit them to an external unit. Each of the radio communication units 273a, 273b receives the radio signal and outputs it to the image combining unit 271.

In the same manner as the image combining unit 246, the image combining unit 271 acquires the left-eye image data and the right-eye image data received by the radio communication units 273a, 273b. The image combining unit 271 combines the acquired left-eye image data and right-eye image data to generate one piece of combined image data. The image combining unit 271 outputs the generated combined image data to the processing device 3.

In the case where image data is transmitted and received between the endoscope 2D and the casing 27A via radio communications as in the present modification, too, the advantage similar to that in the above-described second embodiment may be obtained.

### Third Embodiment

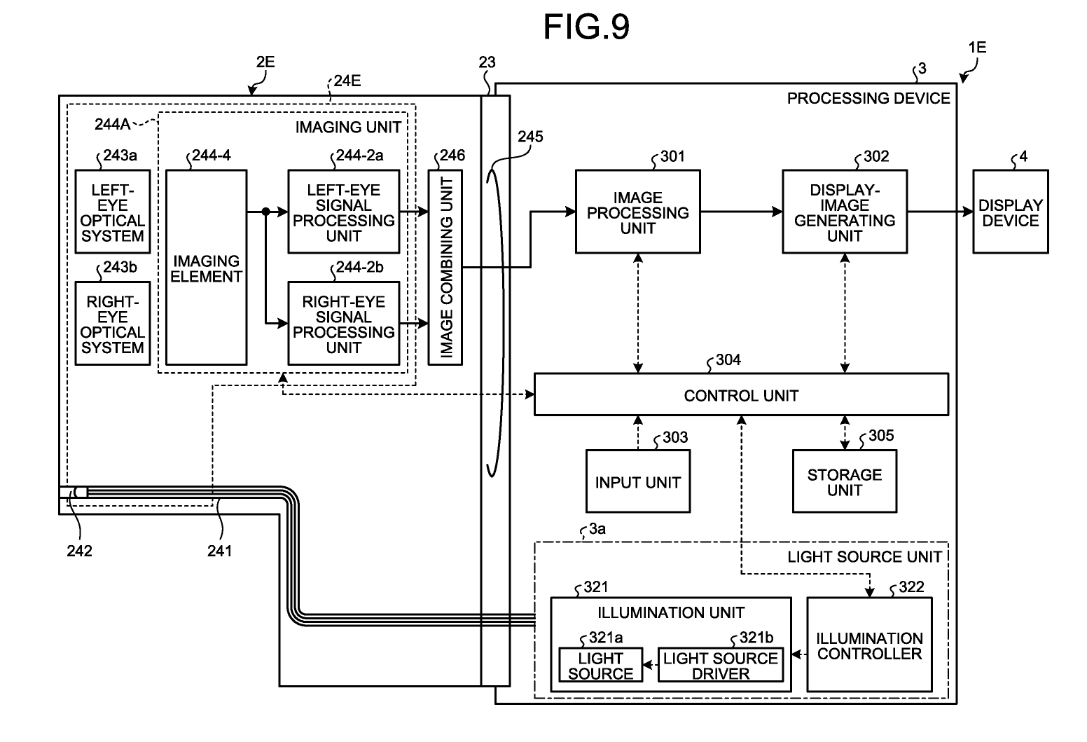

Next, a third embodiment of the disclosure is explained with reference to FIG. 9. According to the third embodiment, right-eye image signals and left-eye image signals are generated by using a single imaging element. FIG. 9 is a block diagram that illustrates a schematic configuration of an endoscope system according to the third embodiment of the disclosure.

An endoscope system 1E illustrated in FIG. 9 includes: an endoscope 2E that captures images inside the body of the subject by inserting the distal end portion into the body of the subject; the processing device 3 that includes the light source unit 3a that generates illumination light to be output from the distal end of the endoscope 2E, executes predetermined signal processing on image signals captured by the endoscope 2E, and controls the overall operation of the endoscope system 1E in an integrated manner; and the display device 4 that displays endoscope images generated during signal processing by the processing device 3. The configuration different from that in the above-described first embodiment is explained below.

The endoscope 2E includes a distal end portion 24E instead of the distal end portion 24 in the above-described configuration of the endoscope 2. The distal end portion 24E includes the light guide 241, the illumination lens 242, the left-eye optical system 243a and the right-eye optical system 243b, an imaging unit 244A, and the image combining unit 246 described above.

The imaging unit 244A includes an imaging element 244-4, the left-eye signal processing unit 244-2a, and the right-eye signal processing unit 244-2b.

In accordance with a control signal received from the processing device 3, the imaging element 244-4 conducts photoelectric conversion on light from each of the left-eye optical system 243a and the right-eye optical system 243b and generates electric signals (image signals) corresponding to one frame forming each of the left-eye image and the right-eye image. Specifically, the imaging element 244-4 has a plurality of pixels arranged in a matrix and has a left-eye image generation area that receives light from the left-eye optical system 243a and a right-eye image generation area that receives light from the right-eye optical system 243b. In the left-eye image generation area and the right-eye image generation area, an image with light from each optical system is formed on a light receiving surface. The imaging element 244-4 sequentially reads electric signals generated by pixels in each area.

The imaging element 244-4 is implemented by using, for example, a CCD image sensor or a CMOS image sensor. Furthermore, the imaging element 244-4 may be configured by using one image sensor, or it may be configured by using a plurality of image sensors, for example, three image sensors.

The left-eye signal processing unit 244-2a performs analog processing to conduct noise removal processing or clamp processing and A/D conversion processing on left-eye image data (analog) output from the imaging element 244-4 and outputs left-eye image data (digital) including the left-eye image to the image combining unit 246.

The right-eye signal processing unit 244-2b performs analog processing to conduct noise removal processing or clamp processing and A/D conversion processing on right-eye image data (analog) output from the imaging element 244-4 and outputs right-eye image data (digital) including the right-eye image to the image combining unit 246.

In the same manner as in the above-described first embodiment, the image combining unit 246 combines the left-eye image data and the right-eye image data to generate one piece of combined image data. The image combining unit 246 outputs the generated combined image data to the processing device 3. In the same manner as in the above-described first embodiment, the processing device 3 generates a disparity image to be presented on the display device 4.

In the case where left-eye image data and right-eye image data are generated based on image signals generated by the single imaging element 244-4, combined image data is generated by the image combining unit 246, and the generated combined image data is transmitted to the processing device 3 as in the third embodiment, too, the advantage similar to that in the above-described first embodiment may be obtained.

### Fourth Embodiment

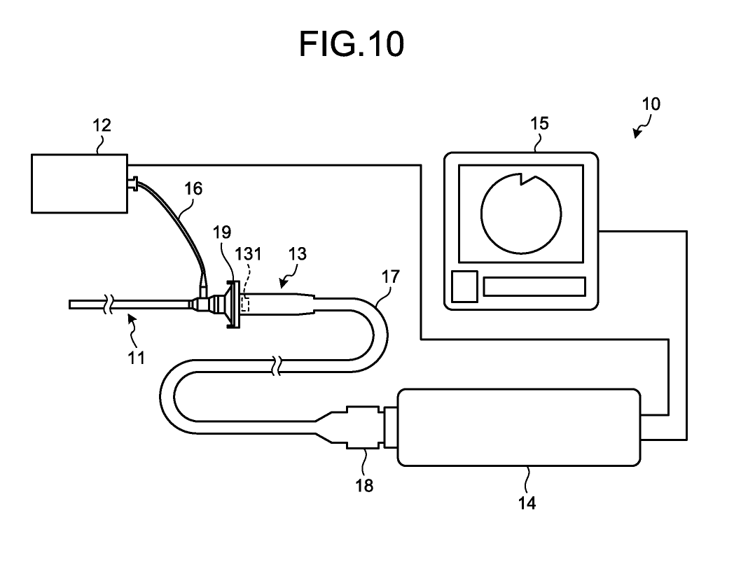

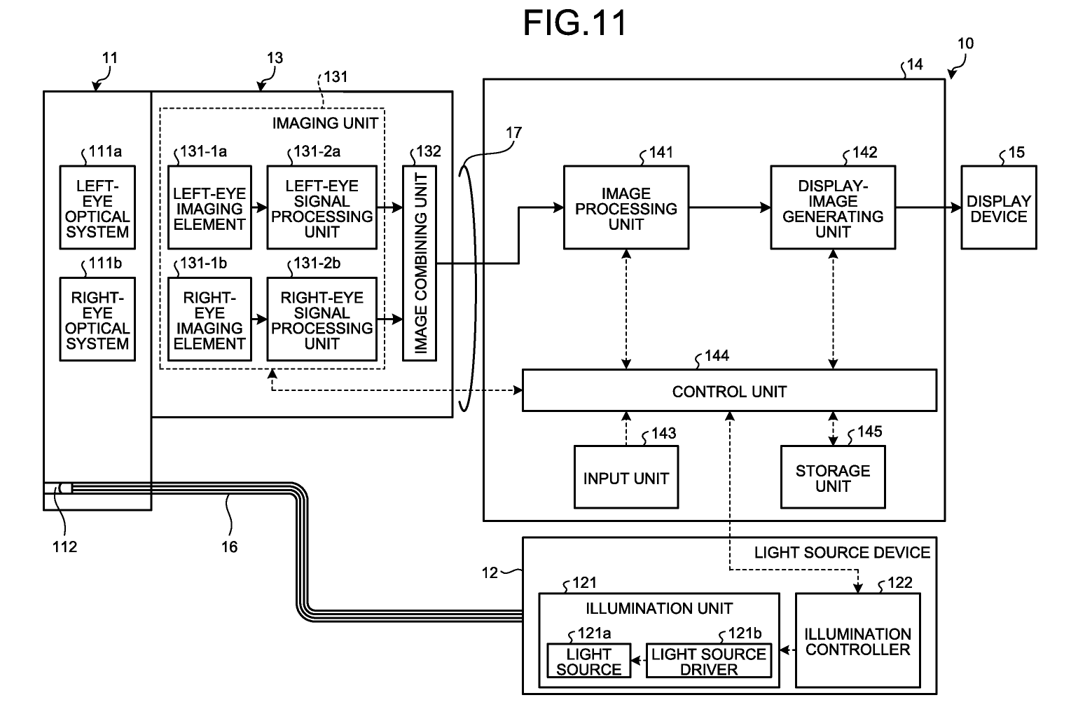

Next, a fourth embodiment of the disclosure is explained with reference to FIG. 10 and FIG. 11. FIG. 10 is a diagram that illustrates a schematic configuration of an endoscope system according to the fourth embodiment of the disclosure. FIG. 11 is a block diagram that illustrates a schematic configuration of an endoscope system according to the fourth embodiment.

An endoscope system 10 illustrated in the drawings includes an insertion portion 11, a light source device 12, a camera head unit 13, a control device 14, a display device 15, a light guide 16, a group cable 17, and a connector 18. The endoscope system 10 is a rigid endoscope used for laparoscopic surgical operation (laparoscope-assisted operation), and the like, by being inserted into the abdominal cavity of the subject.

The insertion portion 11 is rigid and has an elongated shape, and it includes an optical system that collects images of an object with light by being inserted into the body cavity, canal, or the like. Specifically, the insertion portion 11 includes a left-eye optical system 111a and a right-eye optical system 111b for light focus and an illumination lens 112 provided at the distal end of the light guide 16.

The left-eye optical system 111a is configured by using one or more lenses, and it focuses incident light from the object. The left-eye optical system 111a may have an optical zoom function for changing the angle of view and a focus function for changing the focal point.

The right-eye optical system 111b is configured by using one or more lenses, and it focuses incident light from the object with a disparity with respect to the left-eye optical system 111a. The right-eye optical system 111b may have an optical zoom function for changing the angle of view and a focus function for changing the focal point.

The light source device 12 feeds irradiation light to the insertion portion 11 via the light guide 16. The light source device 12 includes an illumination unit 121 and an illumination controller 122. Under the control of the illumination controller 122, the illumination unit 121 outputs illumination light. The illumination unit 121 includes a light source 121a and a light source driver 121b.

In the same manner as the above-described light source 321a, the light source 121a is configured by using an LED light source that outputs white light, one or more lenses, or the like, and outputs light (illumination light) due to driving of the LED light source. The light source 121a is implemented by using any of an LED light source, laser light source, xenon lamp, halogen lamp, and the like. Under the control of the illumination controller 122, the light source driver 121b feeds currents to the light source 121a, thereby causing the light source 121a to output illumination light.

In accordance with a control signal (light control signal) from the control device 14, the illumination controller 122 controls the amount of power supplied to the light source 121a and controls the drive timing of the light source 121a.

The camera head unit 13 is secured to an eye piece portion 19 provided at the proximal end of the insertion portion 11 in an attachable and detachable manner. The camera head unit 13 includes: an imaging unit 131 that receives light focused by the insertion portion 11 (the left-eye optical system 111a and the right-eye optical system 111b), conducts photoelectric conversion into electric signals, and executes predetermined signal processing; and an image combining unit 132 that combines multiple pieces of image data acquired by the imaging unit 131 to generate one piece of combined image data.

The imaging unit 131 includes a left-eye imaging element 131-1a, a right-eye imaging element 131-1b, a left-eye signal processing unit 131-2a, and a right-eye signal processing unit 131-2b.

In accordance with a control signal received from the control device 14, the left-eye imaging element 131-1a conducts photoelectric conversion on light from the left-eye optical system 111a and generates electric signals (left-eye image signals) corresponding to one frame forming one image. For example, the left-eye imaging element 131-1a has the same configuration as that of the above-described left-eye imaging element 244-1a.

In accordance with a control signal received from the control device 14, the right-eye imaging element 131-1b conducts photoelectric conversion on light from the right-eye optical system 111b and generates electric signals (right-eye image signals) corresponding to one frame forming one image. For example, the right-eye imaging element 131-1b has the same configuration as that of the above-described right-eye imaging element 244-1b.

A left-eye image obtained by the left-eye imaging element 131-1a and a right-eye image obtained by the right-eye imaging element 131-1b are images which capture the identical object with different fields of view and which have a disparity.

The left-eye signal processing unit 131-2a performs analog processing for executing noise removal processing or clamp processing or A/D conversion processing on left-eye image data (analog) output from the left-eye imaging element 131-1a and outputs left-eye image data (digital) including a left-eye image to the image combining unit 132.

The right-eye signal processing unit 131-2b performs analog processing for executing noise removal processing or clamp processing or A/D conversion processing on right-eye image data (analog) output from the right-eye imaging element 131-1b and outputs right-eye image data (digital) including a right-eye image to the image combining unit 132.

The image combining unit 132 receives left-eye image data and right-eye image data representing an endoscope image generated by the imaging unit 131. The image combining unit 132 combines the received left-eye image data and right-eye image data to generate one piece of combined image data. The image combining unit 132 outputs the generated combined image data to the control device 14.

The left-eye signal processing unit 131-2a, the right-eye signal processing unit 131-2b, and the image combining unit 132 are configured by using a general-purpose processor such as CPU or a dedicated processor such as various arithmetic circuits to perform a specific function, e.g., ASIC or FPGA.

The control device 14 has a function to perform image processing on images acquired by the camera head unit 13 and has a function to control the overall operation of the endoscope system 10 in an integrated manner. The control device 14 includes an image processing unit 141, a display-image generating unit 142, an input unit 143, a control unit 144, and a storage unit 145.

The image processing unit 141 calculates the pixel value of a luminance component (e.g., the Y component in YCrCb) and the pixel value of each color component, RGB, at each pixel location based on the input combined image and executes signal processing, such as pixel defect correction, optical correction, color correction, optical black subtraction, noise reduction, white balance adjustment, or interpolation processing, on the left-eye image and the right-eye image. Pixel defect correction is to assign the pixel value of a defect pixel based on the pixels values of the surrounding pixels of the defect pixel. Optical correction is to correct optical distortions, or the like, of a lens. Color correction is to correct a color temperature or correct color deviation.

Furthermore, the image processing unit 141 performs zoom processing or enhancement processing on a combined image having undergone the above-described image processing in accordance with the setting input via the input unit 143. Specifically, the image processing unit 141 performs enhancement processing to enhance the R component when, for example, the setting indicating that the red color component is to be enhanced has been made via the input unit 143.

The display-image generating unit 142 generates a synthetic image by synthesizing the background image including the display area of an endoscope image with the textual information regarding the endoscope image. Specifically, the display-image generating unit 142 refers to the storage unit 145 and superimposes the textual information, or the like, regarding the captured endoscope image on the background image, e.g., black background, forming the display screen for synthesizing.

After generating the synthetic image having undergone the above-described synthesis process, the display-image generating unit 142 executes signal processing to obtain signals in a format displayable on the display device 15 and generates image signals for display. Specifically, the display-image generating unit 142 first extracts the left-eye image and the right-eye image of the combined image from the image signals and generates a disparity image, what is called side-by-side image, in which the left-eye image and the right-eye image are located at positions apart from each other and at positions to generate a disparity. Then, the display-image generating unit 142 superimposes the generated disparity image on the image forming the display screen, performs a compression process, or the like, on the image signals including the image, and generates the image signals for display. The display-image generating unit 142 transmits the generated image signals for display to the display device 15. It is possible to use not only side-by-side images but also, for example, line-by-line images that are combined by alternately arranging line data in a left-eye image and line data in a right-eye image while shifting them by the amount of shift to generate a disparity.

The image processing unit 141 and the display-image generating unit 142 are configured by using a general-purpose processor such as CPU or a dedicated processor such as various arithmetic circuits to execute a specific function, e.g., ASIC or FPGA.

The input unit 143 is implemented by using a keyboard, mouse, switch, or touch panel to receive inputs of various signals such as operation command signals for giving commands to operate the endoscope system 10. Furthermore, the input unit 143 may include a switch or a portable terminal such as external tablet-type computer.

The control unit 144 is configured by using a general-purpose processor such as CPU or a dedicated processor such as various arithmetic circuits to perform a specific function, e.g., ASIC, and it controls, for example, driving of each component including the imaging unit 131 and the light source device 12 or controls input/output of information to and from each component. The control unit 144 transmits control information data (e.g., read timing) for capturing control, stored in the storage unit 145, as drive signals to the imaging unit 131 via a predetermined signal line included in the group cable 17. Furthermore, the control unit 144 controls the display device 15 so as to display the image corresponding to image signals for display, generated by the display-image generating unit 142.

The storage unit 145 stores data including various programs for operating the endoscope system 10, various parameters needed to operate the endoscope system 10, and the like, and information regarding the synthesis process, what is called on-screen display (OSD) process, to generate synthetic images by superimposing image information having undergone predetermined image processing on textual information related to the image information. The textual information is information indicating patient information, device information, examination information, or the like.

Furthermore, the storage unit 145 stores various programs including an image-acquisition processing program to implement an image-acquisition processing method of the control device 14. Various programs may be widely distributed by being recorded in a recording medium readable by a computer, such as hard disk, flash memory, CD-ROM, DVD-ROM, or flexible disk. The above-described various programs are available by being downloaded via a communication network. The communication network mentioned here is provided by, for example, the existing public networks, LAN (Local Area Network), or WAN (Wide Area Network) regardless of whether it is wired or wireless.

Furthermore, the storage unit 145 stores information regarding the background image forming a display image and regarding the synthesis process, what is called on-screen display (OSD) process, to generate synthetic images by superimposing textual information related to information on an endoscope image, or the like, on the background image. The textual information is information indicating patient information, device information, examination information, or the like.

The storage unit 145 having the above-described configuration is implemented by using a ROM (Read Only Memory) having previously installed various programs, and the like, a RAM (Random Access Memory) storing calculation parameters, data, and the like, for each process, a hard disk, or the like.

The display device 15 displays images on which the control device 14 has performed image processing. The display device 15 presents a display image that corresponds to image signals received from the control device 14 (the display-image generating unit 142) via a video cable. The display device 15 is configured by using a liquid crystal or organic EL (Electro Luminescence) monitor, or the like.

The light guide 16 is configured by using glass fibers, or the like, and forms a guide path for light emitted by the light source device 12.

The group cable 17 is formed by grouping multiple signal lines; one end thereof is coupled to the camera head unit 13, and the connector 18 is provided at the other end thereof. Signal lines included in the group cable 17 include signal lines for transmitting image signals output from the imaging unit 131 to the control device 14, signal lines for transmitting control signals output from the control device 14 to the imaging unit 131, and the like. The connector 18 is connected to the control device 14 in an attachable and detachable manner. Furthermore, in explanation according to the present embodiment, electric signals are transmitted by using a signal line; however, optical signals may be transmitted.

According to the above-described fourth embodiment of the disclosure, the image combining unit 132 provided in the camera head unit 13 combines the left-eye image data and the right-eye image data representing an endoscope image generated by the imaging unit 131 to generate one piece of combined image data and outputs it to the control device 14. Thus, the control device 14 only has to generate a disparity image for display by executing image processing on a single piece of combined image data; as a result, it is possible to reduce the size of circuitry in the processing device to generate a disparity image by using a right-eye image and a left-eye image.

In the explanation according to the above-described first to fourth embodiments, a left-eye image obtained by the left-eye imaging element and a right-eye image obtained by a right-eye imaging element are images which capture the identical object, in which acquisition areas of an object image are at least partially different from each other, and which have a disparity; however, they may be, for example, images which have a disparity with regard to an identical object, which have the same field of view and have different wavelength bands of illumination light, or which are based on light having passed through filters with different properties. Thus, the endoscope systems 1, 1A to 1E, 10 according to the embodiments make it possible to reduce the size of circuitry in a processing device having a configuration to process images in which the properties of object images are at least partially different from each other. Furthermore, for example, in the case of signal processing on images that are captured by a binocular capsule endoscope and that are images having different fields of view and capturing different objects, i.e., images in which acquisition areas of object images are totally different from each other, it is possible to reduce the size of circuitry in a processing device that processes image signals received from the capsule endoscope.

Furthermore, the explanation according to the above-described first to fourth embodiments uses simultaneous lighting/capturing system in which the light source unit 3a or the light source device 12 emits white illumination light including each color component of RGB and a light receiving unit receives reflected light due to the illumination light; however, for example, it may use sequential lighting/capturing system in which the light source unit 3a individually output light of each color component in sequence and a light receiving unit receives light of each color component.

Furthermore, in explanation according to the above-described first embodiment, the light source unit 3a and the endoscope 2 are configured as separate units; however, a configuration may be such that a light source device is provided in the endoscope 2, for example, a semiconductor light source is provided at the distal end of the endoscope 2. Moreover, the function of the processing device 3 may be assigned to the endoscope 2.

Furthermore, in the explanation according to the above-described first embodiment, the light source unit 3a and the processing device 3 are integrated with each other; however, the light source unit 3a and the processing device 3 may be separate units and the illumination unit 321 and the illumination controller 322 may be provided, for example, outside the processing device 3. Moreover, the light source 321a may be provided at the distal end of the distal end portion 24.

Moreover, the explanation according to the above-described embodiments uses the endoscope systems 1, 1A to 1E using the flexible endoscopes 2, 2A to 2E or the endoscope system 10 including the rigid insertion portion 11 for which the observation target is living tissue, or the like, inside the body of the subject; however, it is also possible to use industrial-use endoscopes that observe the properties of materials, capsule endoscopes, fiberscopes, and endoscope systems using an optical endoscope, such as telescope, having a camera head connected to an eye piece portion thereof.

According to the disclosure, there is an advantage such that it is possible to reduce the size of circuitry in a processing device that generates a disparity image by using a right-eye image and a left-eye image.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope system comprising:
at least one image sensor configured to generate multiple pieces of image data in which acquisition areas of an object image are at least partially different from each other, or multiple pieces of image data having a disparity with regard to an identical object;
a first processor configured to combine the pieces of image data to generate a single piece of combined image data;
a second processor configured to
execute image processing on the combined image data, and
generate display image data to be presented on a display based on the combined image data on which the image processing has been executed, wherein
the second processor is disposed inside a predetermined casing,
the first processor is disposed outside the predetermined casing, and
the combined image data generated by the first processor is transmitted to the predetermined casing.

2. The endoscope system according to claim 1, wherein
the at least one image sensor includes one or more image sensors having a plurality of pixels arranged in a matrix, and
the first processor is configured to generate the combined image data by arranging the pieces of image data along one direction out of directions in which the pixels are arrayed.

3. The endoscope system according to claim 1, wherein
the at least one image sensor includes one or more image sensors having a plurality of pixels arranged in a matrix, and
the first processor is configured to generate the combined image data by periodically arranging the pieces of image data by horizontal line or vertical line of the pixels.

4. The endoscope system according to claim 1, wherein the image sensor and the first processor are disposed in a camera head that is connected to the predetermined casing.

5. The endoscope system according to claim 1, wherein
the image sensor is provided in an insertion portion of an endoscope that is connected to the predetermined casing, and
the first processor is provided in an operating portion of the endoscope.

6. The endoscope system according to claim 1, wherein
the combined image data generated by the first processor is an electric signal,
the endoscope system further comprising:
an electric and optical converter is configured to convert the combined image data combined by the first processor into an optical signal; and
an optical and electric converter is configured to receive the optical signal, convert the received optical signal into an electric signal, and outputs the converted electric signal to the second processor.

7. The endoscope system according to claim 1, further comprising:
a first radio communicator configured to superimpose the combined image data generated by the first processor on a radio signal and transmit the radio signal; and
a second radio communicator configured to receive the radio signal transmitted by the first radio communicator.

8. The endoscope system according to claim 1, further comprising:
a first radio communicator configured to superimpose the pieces of image data generated by the image sensor on a radio signal and transmit the radio signal; and
a second radio communicator that is disposed in a casing different from the predetermined casing and receives the radio signal, wherein
the first processor is configured to generate combined image data based on the radio signal received by the second radio communicator.

9. The endoscope system according to claim 1, wherein the pieces of image data is a digital signal.

10. An endoscope connectable to a processing device including an second processor configured to executes image processing on input image data, and generate a display image to be presented on a display based on the image data on which the image processing has been executed, the endoscope comprising:
at least one image sensor configured to generate multiple pieces of image data in which acquisition areas of an object image are at least partially different from each other, or multiple pieces of image data having a disparity with regard to an identical object; and
a first processor configured to combine the pieces of image data to generate a single piece of combined image data.
